(19) United States
(12) Patent Application Publication
(10) Pub. No.: US 2025/0354071 A1
(43) Pub. Date: Nov. 20, 2025

(54) ACID GAS AND CARBONYL REMOVAL IN A STREAM OF A CHEMICAL PROCESSING PLANT

(71) Applicant: ECOLAB USA Inc., St. Paul, MN (US)

(72) Inventors: Satheesh Kumar SAMBANDAM, Richmond, TX (US); David DIXON, Baton Rouge, LA (US); Stewart PRESNALL, Beaumont, TX (US); Jesse MELLO, Beaumont, TX (US); Keith NAGY, Crown Point, IN (US)

(73) Assignee: ECOLAB USA Inc., St. Paul, MN (US)

(21) Appl. No.: 19/197,220

(22) Filed: May 2, 2025

Related U.S. Application Data
(60) Provisional application No. 63/648,652, filed on May 16, 2024.

(51) Int. Cl. C10G 29/20

(52) U.S. Cl. CPC C10G 29/20; C10G 2300/1025; C10G 2300/107; C10G 2300/1074

(57) ABSTRACT

The present disclosure provides methods, compounds, and compositions for treating industrial streams. A method of treating a stream may include adding a scavenger to the stream and adding an amine to the stream. The stream may include an acid gas and/or a carbonyl-containing compound. The scavenger may include, for example, sodium borohydride, hydroxylamine sulfate, acetaldehyde oxime, sodium cyanoborohydride, or any combination thereof. The amine may include, for example, ethylene diamine, ethyl ethanolamine, diethyl monoethanolamine, or any combination thereof.

### TECHNICAL FIELD

The present disclosure generally relates to inhibiting and/or controlling fouling in industrial systems. More particularly, the disclosure relates to the removal of carbonyl-containing compounds and/or an acid gas from streams included in industrial systems or components of industrial systems.

### BACKGROUND

Pyrolysis is the transformation of a compound into one or more other substances using heat. In the petroleum and petrochemical industries, pyrolysis is useful for the processing of hydrocarbons. This process is often referred to as “cracking.” When the pyrolysis of hydrocarbons is conducted in the presence of steam, it is often referred to as “steam cracking.” The steam cracking of ethane, propane, naphthas, gas oils, and other hydrocarbon feedstocks is a useful process for producing valuable olefins. As a byproduct of the steam cracking process, oxygenated compounds, including carbonyl-containing compounds, are formed. These carbonyl compounds include, but are not limited to, aldehydes and ketones.

Byproducts of hydrocarbon cracking processes include the undesirable acid gases, such as carbon dioxide. Therefore, it is typical for a hydrocarbon cracking plant to have an acid gas removal system to remove acid gasses from the cracked gas. Typically, the acid gas removal system includes passing the gas steam through a basic wash (pH greater than 7) to remove acidic components, including hydrogen sulfide and carbon dioxide gas. In the petroleum and petrochemical industries, unit operations involving basic washes are commonly carried out in equipment referred to as “caustic scrubbers” or “caustic towers.” As an alternative to a caustic tower, about 5% of all ethylene cracking plants use amine, instead of caustic, to remove acid gases. These units are often referred to as “amine scrubbers” or “amine towers.”

In an acid gas removal system, some oxygenated compounds are also removed. It is known in the art of hydrocarbon processing that some of these oxygenated compounds, especially carbonyl compounds and particularly acetaldehyde, will undergo polymerization in the presence of the base. In the acid gas removal system, the acetaldehyde polymer will settle on internal equipment surfaces leading to fouling and eventual plugging. Fouling and plugging of the internal equipment means the unit must be shut down to perform cleaning.

In addition to the caustic scrubbers and caustic towers where treatment is required to inhibit fouling caused by unwanted polymerization, there are other basic wash unit operations in hydrocarbon processing that also require treatment in order to prevent undesirable polymerization of carbonyl compounds. These other basic wash unit operations include, but are not limited to, amine systems to scrub acid gasses, spent caustic oxidizers, and benzene strippers. A spent caustic oxidizer converts sulfides into sulfates to allow caustic to be disposed of, or to be sold. A benzene stripper is a unit that uses hydrocarbon, often a high boiling mixture called “pyrolysis gasoline,” taken from other units in the ethylene plant, to wash “spent caustic.” “Spent caustic” is caustic that has been used in other unit operations, such as the caustic tower. The hydrocarbon “washes” residual benzene out of the spent caustic. The unit is called a benzene stripper because the benzene is stripped from the spent caustic.

### BRIEF SUMMARY

The present disclosure generally relates to inhibiting and/or controlling fouling in industrial systems. In some aspects, the disclosure provides a method of treating a stream comprising adding a scavenger to the stream and adding an amine to the stream.

In certain aspects, the disclosure provides a method of inhibiting formation of an aldol polymer. The method comprises adding a scavenger to a stream comprising a carbonyl-containing compound, adding an amine to the stream, and reacting the carbonyl-containing compound with the scavenger and/or the amine, thereby preventing formation of the aldol polymer, wherein the scavenger and the amine are added to the stream at a weight ratio of about 1:1.

In some embodiments, the present disclosure provides a method of scavenging an acid gas, comprising adding a scavenger to a stream comprising the acid gas, adding an amine to the stream, and reacting the acid gas with the amine and/or the scavenger, thereby scavenging the acid gas, wherein the scavenger and the amine are added to the stream at a weight ratio of about ⅓ scavenger to about ⅔ amine.

The foregoing has outlined rather broadly the features and technical advantages of the present disclosure in order that the detailed description that follows may be better understood. Additional features and advantages of the disclosure will be described hereinafter that form the subject of the claims of this application.

### BRIEF DESCRIPTION OF THE SEVERAL VIEWS OF THE DRAWINGS

A detailed description of the invention is hereafter described with specific reference being made to the drawings in which:

FIG. 1 shows a diagram including examples of addition locations and certain components that may include a stream of the present disclosure;

FIG. 2 shows the aldol polymer reduction performance of three different compositions of the present disclosure;

FIG. 3 shows the results of an evaluation of ethylene diamine under and not under CO₂ bubbling conditions; and

FIG. 4 shows the results of an evaluation of hydroxyl amine sulfate under and not under CO₂ bubbling conditions.

### DETAILED DESCRIPTION

Various embodiments are described below. The relationship and functioning of the various elements of the embodiments will be better understood in light of the following detailed description. However, elements and embodiments are not strictly limited to those explicitly described below.

Examples of methods and materials are described below, although methods and materials similar or equivalent to those described herein can be used in practice or testing of the present disclosure. All publications, patent applications, patents and other reference materials mentioned herein are incorporated by reference in their entirety. The materials, methods, and examples disclosed herein are illustrative only and not intended to be limiting.

Unless otherwise defined, all technical and scientific terms used herein have the same meaning as commonly understood by one of ordinary skill in the art. In case of conflict, the present document, including definitions, will control.

The terms “polymer,” “copolymer,” “polymerize,” “copolymerize,” and the like include not only polymers comprising two monomer residues and polymerization of two different monomers together, but also include (co) polymers comprising more than two monomer residues and polymerizing together more than two or more other monomers. For example, a polymer as disclosed herein includes a terpolymer, a tetrapolymer, polymers comprising more than four different monomers, as well as polymers comprising, consisting of, or consisting essentially of two different monomer residues. Additionally, a “polymer” as disclosed herein may also include a homopolymer, which is a polymer comprising a single type of monomer unit.

Unless specified differently, the polymers of the present disclosure may be linear, branched, crosslinked, structured, synthetic, semi-synthetic, natural, and/or functionally modified. A polymer of the present disclosure can be in the form of a solution, a dry powder, a liquid, or a dispersion, for example.

The present disclosure provides methods, compounds, and compositions for improving acid gas, such as carbon dioxide (CO₂), and carbonyl removal in various locations, such as acid gas removal units. Compounds and compositions of the present disclosure may include a scavenger and an amine-containing compound (which may be interchangeably referred to herein as an “amine”).

Illustrative, non-limiting examples of scavengers include sodium borohydride, hydroxylamine sulfate, acetaldehyde oxime, sodium cyanoborohydride, and any combination thereof.

Illustrative, non-limiting examples of amines include ethylene diamine, ethyl ethanolamine, diethyl monoethanolamine, and any combination thereof. In some embodiments, the amine excludes a substituted hydroxylamine.

The methods of the present disclosure utilize the presently disclosed scavengers and amines to treat various streams comprising a carbonyl-containing compound (which may be interchangeably referred to herein as a “carbonyl compound”) and/or CO₂. In accordance with the methods disclosed herein, the scavenger is added to the stream and the amine is added to the stream. The scavenger may be added to the stream before, after, and/or with the amine. The amine may be added to the stream before, after, and/or with the scavenger.

The amine and the scavenger may be added to the stream at a single location or at multiple locations. For example, the amine may be added at a first location and the scavenger may be added at a second location, which may be the same location or a different location than the first location. The amine and the scavenger may be added together in a single composition and/or they may be added separately. The amine and the scavenger may be added automatically and/or manually, continuously, or intermittently.

The amine may be added at any number of locations, such as a first location, a second location, and/or a third location. The scavenger may also be added at any number of locations, such as a first location, a second location, and/or a third location. The “first location” for the amine may be the same location as the “first location” for the scavenger or it may be different. The “second location” for the amine may be the same location as the “second location” for the scavenger or it may be different. The “third location” for the amine may be the same location as the “third location” for the scavenger or it may be different. Any of the foregoing locations may comprise a stream and the stream may include the carbonyl-containing compound and/or the CO₂.

The one or more locations where the compounds and/or compositions are added may be found in, for example, a caustic tower, an amine gas scrubber, a spent caustic oxidizer, a caustic scrubber, and/or a benzene stripper. Any of these industrial components may comprise the stream containing the carbonyl compound and/or the CO₂.

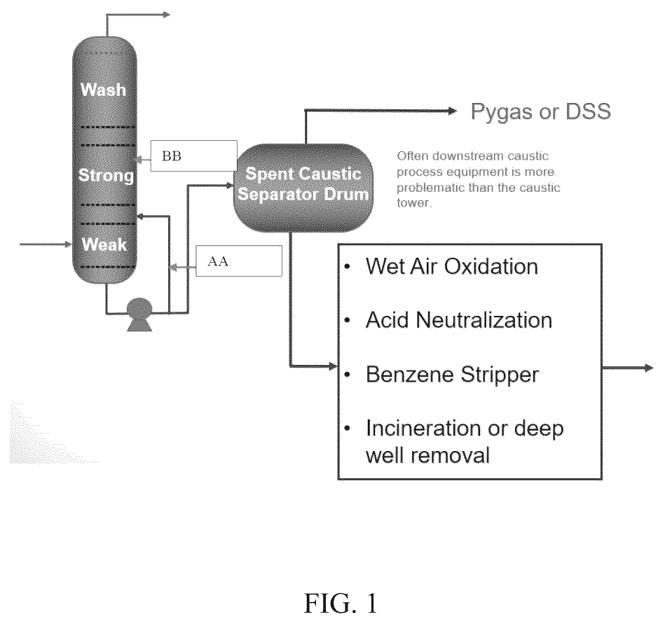

In accordance with FIG. 1, a caustic tower is shown having a “wash” section, a “strong” section, and a “weak” section. As an illustrative, non-limiting example, the scavenger may be added to the strong section of the caustic tower (such as at location “BB”), a weak section of the caustic tower (such as at location “AA”), before the caustic tower, after the caustic tower, such as to a spent caustic separator drum, a benzene stripper, etc., and any combination thereof.

In accordance with the present disclosure, the strong section of the caustic tower has a caustic strength, for example, about 8-12% and the weak section has a caustic strength of about 0.5-4%. Some towers have additional sections, such as four sections, which may include a water wash section and a mid-section, which may have a caustic strength of, for example, about 4-8%. The scavenger, amine, and/or any other compounds or compositions disclosed herein may be added to any section, together or independently, in any order.

For example, the amine may be added to the strong section of the caustic tower, the weak section of the caustic tower, before the caustic tower, after the caustic tower, such as to a spent caustic separator drum, a benzene stripper, etc., and any combination thereof.

As illustrative, non-limiting examples, the scavenger may be added to the wash section of the caustic tower, the strong section of the caustic tower, the weak section of the caustic tower, the spent caustic separator drum, the benzene stripper, an amine gas scrubber, a spent caustic oxidizer, a caustic scrubber, a pipeline connecting any of the foregoing components, and any combination of the foregoing.

As illustrative, non-limiting examples, the amine may be added to the wash section of the caustic tower, the strong section of the caustic tower, the weak section of the caustic tower, the spent caustic separator drum, the benzene stripper, an amine gas scrubber, a spent caustic oxidizer, a caustic scrubber, a pipeline connecting any of the foregoing components, and any combination of the foregoing.

For example, in some embodiments, the scavenger may be added to a strong section of the caustic tower and the amine may be added to a weak section of the caustic tower. In some embodiments, the scavenger may be added to a weak section of the caustic tower and the amine may be added to a strong section of the caustic tower. In certain embodiments, the scavenger may be added to a strong section and/or a weak section of the caustic tower and the amine may be added to the strong section and/or the weak section of the caustic tower. Additionally or alternatively, the scavenger and/or amine may be added to a pipeline upstream of and in communication with the caustic tower.

Any of the foregoing locations or components of industrial systems may include the stream comprising the carbonyl compound and/or the CO₂. Illustrative, non-limiting examples of streams include liquefied petroleum gas, natural gas, a naphtha, an atmospheric gas oil, a vacuum gas oil, a condensate, a hydrocarbon, ethane, ethylene, propane, a vinyl acetate monomer, a cracked gas stream, such as a pyrolysis cracked gas stream, and any combination thereof.

The stream to be treated with the amine and/or the scavenger may include any amount of the carbonyl compound and the CO₂.

For example, the stream may comprise from about 0 ppm to about 10,000 ppm of the CO₂, such as from about 1 ppm to about 8,000 ppm, about 1 ppm to about 6,000 ppm, about 1 ppm to about 4,000 ppm, about 1 ppm to about 2,000 ppm, about 1 ppm to about 1,000 ppm, about 1 ppm to about 800 ppm, about 1 ppm to about 600 ppm, about 1 ppm to about 400 ppm, about 1 ppm to about 200 ppm, about 1 ppm to about 100 ppm, about 1 ppm to about 50 ppm, about 1 ppm to about 25 ppm, about 1 ppm to about 10 ppm, about 1 ppm to about 5 ppm, about 0.1 ppm to about 5 ppm, about 0 ppm to about 10 ppm, about 0 ppm to about 5 ppm, about 0 ppm to about 1 ppm, about 10 ppm to about 400 ppm, about 50 ppm to about 400 ppm, about 100 ppm to about 400 ppm, about 100 ppm to about 300 ppm, about 100 ppm to about 200 ppm, or about 125 ppm to about 175 ppm of the CO₂. The amounts listed in this paragraph may refer to a stream before addition of the amine and/or scavenger or the amounts listed in this paragraph may refer to a stream that has been treated with the amine and/or scavenger.

The stream may comprise, for example, from about 0 ppm to about 10,000 ppm of the carbonyl compound, such as from about 1 ppm to about 8,000 ppm, about 1 ppm to about 6,000 ppm, about 1 ppm to about 4,000 ppm, about 1 ppm to about 2,000 ppm, about 1 ppm to about 1,000 ppm, about 1 ppm to about 800 ppm, about 1 ppm to about 600 ppm, about 1 ppm to about 400 ppm, about 1 ppm to about 200 ppm, about 1 ppm to about 100 ppm, about 1 ppm to about 50 ppm, about 1 ppm to about 25 ppm, about 1 ppm to about 10 ppm, about 1 ppm to about 5 ppm, about 0.1 ppm to about 5 ppm, about 0 ppm to about 10 ppm, about 0 ppm to about 5 ppm, about 0 ppm to about 1 ppm, about 10 ppm to about 500 ppm, about 50 ppm to about 500 ppm, about 100 ppm to about 500 ppm, about 100 ppm to about 400 ppm, about 100 ppm to about 300 ppm, or about 100 ppm to about 200 ppm of the carbonyl compound. The amounts listed in this paragraph may refer to a stream before addition of the amine and/or scavenger or the amounts listed in this paragraph may refer to a stream that has been treated with the amine and/or scavenger.

The streams contemplated by the present disclosure may include various carbonyl compounds. Illustrative, non-limiting examples of carbonyl compounds include acetaldehyde, acetone, and any combination thereof.

In various streams, if left alone, the carbonyl compound will react and form an undesired aldol polymer, which may lead to fouling of the industrial system (and/or component of the industrial system) comprising the stream. In some embodiments, the amine reacts with the carbonyl compound and prevents, inhibits, and/or reduces formation of the aldol polymer. In some embodiments, the scavenger reacts with the carbonyl compound and prevents, inhibits, and/or reduces formation of the aldol polymer.

For example, the scavenger may react with the carbonyl compound and prevent formation of the aldol polymer. Additionally or alternatively, the amine may react with the carbonyl compound and prevent formation of N-acetyl ethylenediamine when the amine comprises ethylene diamine. Additionally or alternatively, the amine may react with the CO₂.

The scavenger and the amine may be added to the stream at various weight ratios depending upon the needs of the industrial system. For example, a method of the present disclosure may include adding the scavenger and the amine to the stream at a weight ratio of about 2:3 to about 3:2, such as from about 2.5:3 to about 3:2 or about 2:3 to about 2.5:2, scavenger to amine.

However, a method of the present disclosure may comprise adding the scavenger and the amine to the stream at a weight ratio of about 1:4 to about 1:2, such as about 1:3, scavenger to amine.

Under certain circumstances, the weight ratio of scavenger to amine may be selected based upon the amount of carbonyl compound and/or CO₂ in the stream. The weight ratio of scavenger to amine may also be selected based upon the desired outcome, e.g., carbonyl compound removal from the stream (prevention/inhibition of aldol polymer formation), CO₂ removal from the stream, or a combination thereof.

For example, to effectively prevent, inhibit, and/or reduce formation of the aldol polymer, a weight ratio of about 1:1 scavenger to amine may be used. To effectively reduce spent caustic, e.g., by removal of CO₂ from the stream, a weight ratio of about ⅓ scavenger to about ⅔ amine may be used.

As an illustrative, non-limiting example, the amount of scavenger to be added to the stream may be about 0 to about 4 times the carbonyl concentration in the stream, such as about 1 times, about 1.5 times, about 2 times, about 2.5 times, about 3 times, or about 3.5 times the concentration of the carbonyl compound in the stream.

In terms of ppm, the amount of scavenger added to the stream may be from about 0 ppm to about 1,000 ppm, such as about 1 ppm to about 800 ppm, about 1 ppm to about 600 ppm, about 1 ppm to about 400 ppm, about 1 ppm to about 200 ppm, about 50 ppm to about 500 ppm, about 50 ppm to about 300 ppm, or about 100 ppm to about 400 ppm.

As an additional illustrative, non-limiting example, the amount of amine to be added to the stream may be about 0 to about 4 times the CO₂ concentration in the stream, such as about 1 times, about 1.5 times, about 2 times, about 2.5 times, about 3 times, or about 3.5 times the concentration of CO₂ in the stream.

In terms of ppm, the amount of amine added to the stream may be from about 0 ppm to about 1,000 ppm, such as about 1 ppm to about 800 ppm, about 1 ppm to about 600 ppm, about 1 ppm to about 400 ppm, about 1 ppm to about 200 ppm, about 50 ppm to about 500 ppm, about 50 ppm to about 300 ppm, or about 100 ppm to about 400 ppm.

In certain aspects, the present disclosure provides a method of inhibiting formation of an aldol polymer. The method comprises adding a scavenger to a stream comprising a carbonyl-containing compound, adding an amine to the stream, and reacting the carbonyl-containing compound with the scavenger and optionally the amine, thereby preventing formation of the aldol polymer, wherein the scavenger and the amine are added to the stream at a weight ratio of about 1:1. The stream may also comprise the acid gas, such as carbon dioxide, in any amount, such as about 1 ppm to about 1,000 ppm, about 100 ppm to about 1,000 ppm, about 100 ppm to about 200 ppm, about 300 ppm to about 1,000 ppm, about 500 ppm to about 1,000 ppm, or about 100 to about 300 ppm. In some embodiments, a caustic tower comprises the stream. The amine may be added to a strong section of the caustic tower, among other locations throughout the process, and the scavenger may be added to a weak section of the caustic tower, among other locations.

In certain aspects, the present disclosure provides a method of scavenging carbon dioxide thereby reducing spent caustic. The method comprises adding a scavenger to a stream comprising the carbon dioxide, adding an amine to the stream, and reacting the carbon dioxide with the amine and optionally the scavenger, thereby scavenging the carbon dioxide. The scavenger and the amine may be added to the stream at a weight ratio of about ⅓ scavenger to about ⅔ amine. The stream may also comprise a carbonyl-containing compound. The amine may be added to a weak section of the caustic tower, among other locations, and the scavenger may be added to a strong section of the caustic tower, among other locations.

The technology disclosed herein is effective for removal of acid gasses besides carbon dioxide, such as carbonyl sulfide and hydrogen sulfide. The present disclosure therefore provides a method of scavenging an acid gas. The method comprises adding a scavenger to a stream comprising the acid gas, adding an amine to the stream, and reacting the acid gas with the amine and optionally the scavenger, thereby scavenging the acid gas. The scavenger and the amine may be added to the stream at a weight ratio of about ⅓ scavenger to about ⅔ amine. The stream may also comprise a carbonyl-containing compound. The amine may be added to a weak section of the caustic tower, among other locations, and the scavenger may be added to a strong section of the caustic tower, among other locations.

The foregoing may be better understood by reference to the following examples, which are intended for illustrative purposes and are not intended to limit the scope of the disclosure or its application in any way.

### EXAMPLES

A semi-batch experiment with vinyl acetate monomer flowing through a syringe pump into a flask containing 2M caustic dosed with carbonyl scavenger was conducted. The objective was to sample the reaction vessel at different time intervals to track the progress of the reaction under inhibited and uninhibited conditions. Aldol polymer concentration in ppm was measured using an evaporative light scattering detector (ELSD) and the experiment was stopped after 30 min to get at least 5 data points to plot the reaction profile.

An ELSD was used to measure the amount of polymer in the samples but the caustic present in the reaction medium remains even after solvent evaporation so it will show up along with the polymer during detection. To address this issue, the reaction mixture of caustic and aldol polymer sampled at different time intervals was added to a vial containing a solvent, e.g., tetrahydrofuran, that can dissolve the aldol polymer. The resultant solvent was then injected in to the ELSD, which helped quantify the aldol polymer present in the sample.

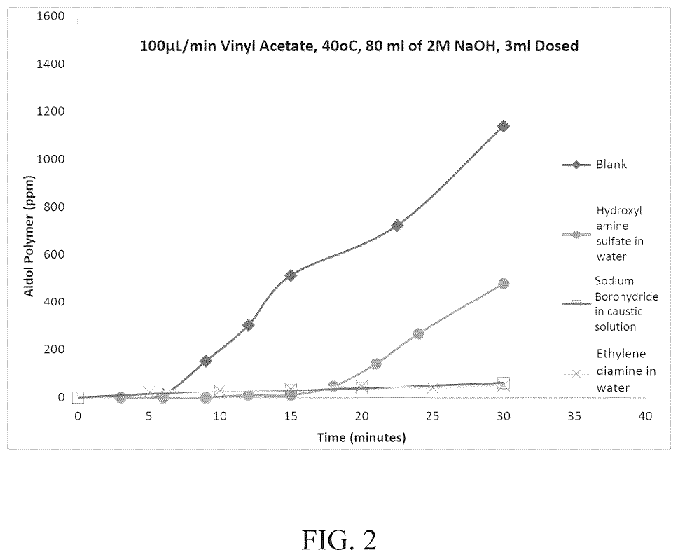

FIG. 2 shows the aldol polymer reduction performance of three different inventive compositions of the present disclosure including hydroxyl amine sulfate in water, sodium borohydride in caustic solution, and ethylene diamine in water. As can be seen, the amount of aldol polymer formed was greatest when no composition was added (blank) and lowest when ethylene diamine in water was added.

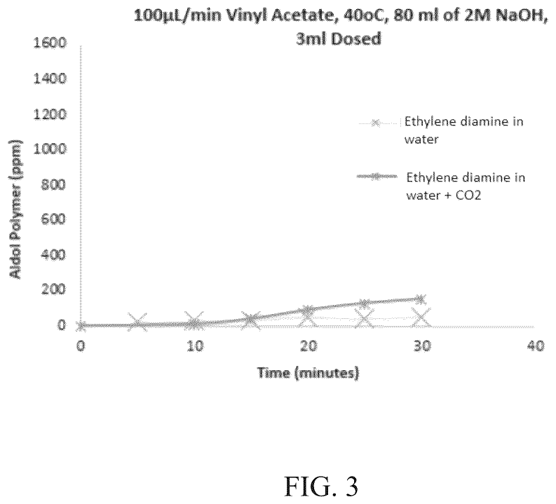

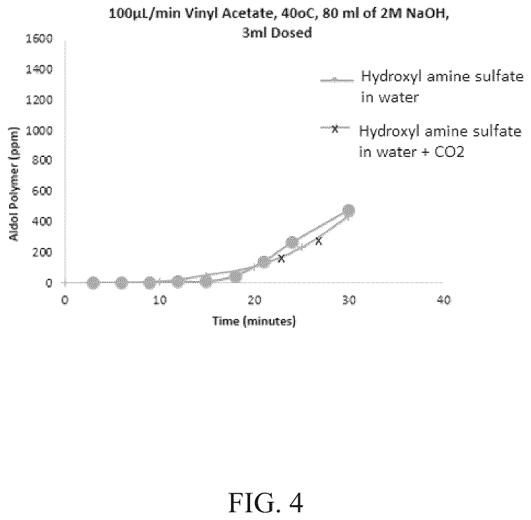

Performance evaluation of inventive compositions of the present disclosure under CO₂ bubbling conditions was also tested. As can be seen in FIG. 3, ethylene diamine in water shows a marginal drop in performance under the presence of CO₂. As can be seen in FIG. 4, the performance of hydroxyl amine sulfate in water was not impacted by the presence of CO₂. While CO₂ reaction with caustic is preferred rather than with the amine, the data shows that ethylene diamine is consumed by CO₂, leading to its drop in performance.

Examination of the solution of aliquots sampled during ethylene diamine performance testing under CO₂ bubbling conditions (100 ml and 200 ml/min), solids were seen at the bottom of the vial. These solids were then collected, analyzed, and identified as N-acetyl ethylenediamine, sometimes further including one or more urea-like compounds.

In additional experiments, a laboratory gas scrubber will be used to evaluate the efficiency of various inventive compositions of the present disclosure in CO₂ and H₂S removal from a gas stream containing carbonyls passing through a caustic scrubber that simulates a caustic tower in an ethylene plant.

In a first set of experiments, an inlet stream including nitrogen (carrier gas) and CO₂ along with acetaldehyde will be heated (boiling point ˜20° C.) to mix with the carrier gas. The inlet stream will be mixed from a custom mixed gas cylinder with nitrogen and CO₂ (about 50 ppm, about 250 ppm, and about 500 ppm). This gas will be mixed with a carbonyl concentration of about 100 ppm before entering the scrubber. When the mixture is passed through the circulating 6% caustic solution containing ethylene diamine in water varied between about 10 ppm to about 5,000 ppm, one can calculate the ethylene diamine consumption (by IC) both in the presence and absence of CO₂ for a fixed carbonyl concentration of 100 ppm. The same experiment can be repeated by adding other chemistries, such as sodium borohydride in caustic solution or any other carbonyl scavenger, to see the benefit of having both ethylene diamine in water and a carbonyl scavenger. To simulate hydrocarbon solvent present in the caustic tower, toluene or pyrolysis gasoline may be added to caustic solution. The foregoing procedure can be carried out with acid gasses other than CO₂, such as H₂S.

All of the compositions and methods disclosed and claimed herein can be made and executed without undue experimentation in light of the present disclosure. While this invention may be embodied in many different forms, there are described in detail herein specific preferred embodiments of the invention. The present disclosure is an exemplification of the principles of the invention and is not intended to limit the invention to the particular embodiments illustrated. In addition, unless expressly stated to the contrary, use of the term “a” is intended to include “at least one” or “one or more.” For example, “a scavenger” is intended to include “at least one scavenger” or “one or more scavengers.”

Any ranges given either in absolute terms or in approximate terms are intended to encompass both, and any definitions used herein are intended to be clarifying and not limiting. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges (including all fractional and whole values) subsumed therein.

Any composition disclosed herein may comprise, consist of, or consist essentially of any element, component and/or ingredient disclosed herein or any combination of two or more of the elements, components or ingredients disclosed herein.

Any method disclosed herein may comprise, consist of, or consist essentially of any method step disclosed herein or any combination of two or more of the method steps disclosed herein.

The transitional phrase “comprising,” which is synonymous with “including,” “containing,” or “characterized by,” is inclusive or open-ended and does not exclude additional, un-recited elements, components, ingredients and/or method steps.

The transitional phrase “consisting of” excludes any element, component, ingredient, and/or method step not specified in the claim.

The transitional phrase “consisting essentially of” limits the scope of a claim to the specified elements, components, ingredients and/or steps, as well as those that do not materially affect the basic and novel characteristic(s) of the claimed invention.

Unless specified otherwise, all molecular weights referred to herein are weight average molecular weights and all viscosities were measured at 25° C. with neat (not diluted) polymers.

As used herein, the term “about” refers to the cited value being within the errors arising from the standard deviation found in their respective testing measurements, and if those errors cannot be determined, then “about” may refer to, for example, within 5%, 4%, 3%, 2%, or 1% of the cited value.

Furthermore, the invention encompasses any and all possible combinations of some or all of the various embodiments described herein. It should also be understood that various changes and modifications to the presently preferred embodiments described herein will be apparent to those skilled in the art. Such changes and modifications can be made without departing from the spirit and scope of the invention and without diminishing its intended advantages. It is therefore intended that such changes and modifications be covered by the appended claims.

What is claimed is:

1. A method of treating a stream, comprising:
adding a scavenger to the stream, and
adding an amine to the stream.

2. The method of claim 1, wherein the scavenger is selected from the group consisting of sodium borohydride, hydroxylamine sulfate, acetaldehyde oxime, sodium cyanoborohydride, and any combination thereof.

3. The method of claim 1, wherein the amine is selected from the group consisting of ethylene diamine, ethyl ethanolamine, diethyl monoethanolamine, and any combination thereof.

4. The method of claim 1, wherein the stream comprises a carbonyl-containing compound.

5. The method of claim 4, wherein the carbonyl-containing compound is selected from the group consisting of acetaldehyde, acetone, and any combination thereof.

6. The method of claim 4, further comprising reacting the carbonyl-containing compound with the scavenger and preventing formation of an aldol polymer.

7. The method of claim 4, further comprising adding the scavenger and the amine to the stream at a weight ratio of about 2:3 to about 3:2 scavenger to amine.

8. The method of claim 4, further comprising reacting the carbonyl-containing compound with the amine, wherein the amine comprises ethylene diamine, and preventing formation of N-acetyl ethylenediamine.

9. The method of claim 1, wherein the stream comprises an acid gas.

10. The method of claim 9, further comprising reacting the acid gas with the amine.

11. The method of claim 9, further comprising adding the scavenger and the amine to the stream at a weight ratio of about 1:4 to about 1:2 scavenger to amine.

12. The method of claim 1, further comprising adding the scavenger to the stream before, after, and/or with the amine.

13. The method of claim 1, wherein a caustic tower, an amine gas scrubber, a spent caustic oxidizer, a caustic scrubber, and/or a benzene stripper comprises the stream.

14. The method of claim 13, further comprising adding the scavenger and/or the amine to a strong section of the caustic tower, a weak section of the caustic tower, before the caustic tower, after the caustic tower, and any combination thereof.

15. The method of claim 1, wherein the stream comprises liquefied petroleum gas, natural gas, a naphtha, an atmospheric gas oil, a vacuum gas oil, a condensate, a hydrocarbon, ethane, ethylene, propane, a vinyl acetate monomer, a pyrolysis cracked gas stream, and any combination thereof.

16. The method of claim 1, wherein the amine excludes a substituted hydroxylamine.

17. A method of inhibiting formation of an aldol polymer, comprising:
adding a scavenger to a stream comprising a carbonyl-containing compound,
adding an amine to the stream, and
reacting the carbonyl-containing compound with the scavenger and/or the amine, thereby preventing formation of the aldol polymer, wherein the scavenger and the amine are added to the stream at a weight ratio of about 1:1.

18. The method of claim 17, wherein the stream comprises an acid gas.

19. The method of claim 17, wherein a caustic tower comprises the stream, the amine is added to a strong section of the caustic tower and the scavenger is added to a weak section of the caustic tower.

20. A method of scavenging an acid gas, comprising:
adding a scavenger to a stream comprising the acid gas,
adding an amine to the stream, and
reacting the acid gas with the amine and/or the scavenger, thereby scavenging the acid gas, wherein the scavenger and the amine are added to the stream at a weight ratio of about ⅓ scavenger to about ⅔ amine.
